(19) United States
(12) Patent Application Publication
(10) Pub. No.: US 2010/0233350 A1
(43) Pub. Date: Sep. 16, 2010

(54) Drug delivery composition and methods of making same using nanofabrication

(75) Inventor: Robert Herrmann, Boston, MA (US)

Correspondence Address: KENYON & KENYON LLP, 1500 K STREET N.W., SUITE 700, WASHINGTON, DC, 20005, US

(73) Assignee: Boston Scientific Scimed, Inc.

(21) Appl. No.: 11/375,021

(22) Filed: Mar. 15, 2006

(51) Int. Cl. A61K 9/52

(52) U.S. Cl. 427/2.21

(57) ABSTRACT

The present invention relates to drug delivery compositions, drug delivery units, drug delivery devices and methods of making the same using nanofabrication processes. The nanofabrication processes are used to make pores in a matrix. Exemplary nanofabrication processes include nanoimprinting, reverse imprinting, nanolithography, and photolithography.

### TECHNICAL FIELD

The present invention relates to drug delivery compositions, drug delivery units, drug delivery devices and methods of making the same using nanofabrication processes.

### BACKGROUND

Many therapeutic agents are more effective if delivered to a patient as a time-release injection or on the surface of an implantable medical device. Thus, there is increasing interest in drug delivery systems that allow for a therapeutic agent to be delivered into a patient, while still providing a sustained release rate.

One traditional method of achieving time-release therapeutic agents in this form is to manufacture microspheres containing the therapeutic agent. Generally, a bulk lot of microspheres is fabricated. The microspheres may comprise a variety of materials, for example a polymer with pores to contain the therapeutic agent. When the microspheres are formed, the processing conditions determine the pore structure. Thus, with prior manufacturing techniques, pore structure can generally only be determined for a bulk lot of microspheres as a whole. For example, the average pore size for a particular lot of microspheres may be known, but specific pore sizes for a particular microsphere or group of microspheres may not be known. In some methods, therapeutic agent may be disposed unevenly throughout the microspheres, leading to unpredictable or undesirable release kinetics.

These methods may cause microsphere delivery systems to be unsuitable for some purposes. For example, therapeutic agents requiring precise release rates may not be suitable for use in microspheres made by prior manufacturing techniques, since only the bulk pore structure is known. Similarly, use of such microspheres may result in a “burst effect,” whereby a large quantity or percentage of the therapeutic agent is released immediately on introduction of the microspheres to the intended delivery site. In some cases, this effect may cause a therapeutic agent to be released at toxic or otherwise harmful concentrations, rendering such microsphere delivery systems unsuitable for use. When used with other therapeutic agents, or when different pore sizes are created during the manufacturing process, release rates may vary during the treatment. This may cause such microsphere delivery systems to be undesirable for therapeutic agents requiring a constant, steady release rate.

Microspheres may also be undesirable due to their bulk material properties, herein referred to as “shape performance”. For example, where it is desirable that the delivery system occlude a vessel, orifice, or puncture, microspheres may be unsuitable. Since microspheres are generally roughly spherical, they may flow easily through openings or vessels, and be unsuitable for applications where occlusion of the opening or vessel is desired.

### SUMMARY OF THE INVENTION

In an embodiment, the present invention provides a method of manufacturing a drug delivery composition. The method comprises the steps of providing a first layer of matrix material and forming pores in the first layer of matrix material. The method further comprises providing a second layer of matrix material on the first layer of matrix material and forming pores in the second layer of matrix material. The pores may be formed in the second layer after or prior to the second layer being placed on the first layer. The method further comprises placing a therapeutic agent in the pores of the first and second layers of matrix material. The pores may be formed such that they have a square or rectangular cross section. Such shapes may allow for pre-determined release rates to be easily implemented in the composition. It will be understood that “release rate” refers to the amount of therapeutic delivered over a period of time per drug delivery unit under physiologic conditions. The therapeutic agent may be placed in the pores prior to or after the second layer is placed on the first layer. Additional layers may be placed on the first and second layers such that a complex pore structure is built up by the layering of relatively simple pore structures.

In an embodiment, the present invention provides a method of manufacturing a plurality of drug delivery units comprising determining a desired release rate, forming a lattice comprising matrix material and therapeutic agents, and separating the lattice into drug delivery units. The lattice spacing, the number of drug delivery units, or both are based on the desired release rate.

In an embodiment, the present invention provides a method of manufacturing a drug delivery composition comprising determining desired release kinetics and forming a lattice. The lattice is formed by depositing a plurality of layers of matrix material and forming pores in at least one of the layers of matrix material. The pores may be formed after or prior to each layer being placed on the previous layer. The method further comprises placing therapeutic agents in the pores, wherein the lattice spacing is based on the desired release kinetics. The therapeutic agent may be placed in the pores prior to or after each layer is placed on the previous layer.

In an embodiment, the present invention provides a method of manufacturing a drug delivery device comprising providing a medical device body and depositing a first layer of matrix material having pores with rectangular cross-sections on the medical device body. The method further comprises depositing therapeutic agents in the pores of the first layer of matrix material. The pores may be formed in each layer prior to or after the layer is placed on the previous layer. The therapeutic agent may be placed in the pores prior to or after each layer is placed on the previous layer.

In an embodiment, the present invention provides a medical device comprising a medical device body and a plurality of layers of matrix material disposed on the medical device body. Each layer has pores with rectangular cross-sections. The medical device further comprises a therapeutic agent that is disposed in the pores of each layer of matrix material.

### BRIEF DESCRIPTION OF THE DRAWINGS

FIG. 1 shows a side view of a nanofabrication process to manufacture a drug delivery composition and drug delivery unit.

FIG. 2 shows a side view of a reverse imprinting process to form pores in a matrix material.

FIG. 3 shows a side view of a nanoimprinting process to form pores in a matrix material.

FIG. 4 shows side view of a nanolithography process to form pores in a matrix material.

FIG. 5 is an exploded perspective view of a drug delivery unit.

### DETAILED DESCRIPTION OF THE INVENTION

Embodiments of the present invention provide drug delivery compositions, drug delivery units and drug delivery devices as well as methods of making the same using nanofabrication processes. Such nanofabrication processes can be any of the several methods of nanofabrication which remove material from a layer of material and which are capable of forming pores as small as about 100 to 200 nanometers (nm). Larger pore sizes may be desirable for various applications. Nanofabrication processes include, for example, nanolithography, nanoimprinting, reverse imprinting, and soft lithography. Several examples of such processes are discussed herein, but other methods and variations are also possible. Such nanofabrication processes generally include providing a layer of matrix material and forming pores in the matrix material. The matrix material can be provided by depositing the matrix material on a substrate, for example. After pores are formed, therapeutic agents are placed in the pores. The configuration of the pores may be based on the release rate and kinetics desired for the therapeutic agent. For example, in certain embodiments, it may be preferable for the pores to be rectangular in order for the therapeutic agent to have certain release rates and kinetics. Additional layers comprising matrix material and therapeutic agent may be deposited on the initial layer by repeating the process of depositing a layer of matrix material and forming pores in the matrix material. The layers may each be deposited using the same nanolithographic technique, or different techniques may be used. Therapeutic agent may be placed in the pores of each layer prior to depositing the following layer, or it may be placed into all pores in the layered structure after all layers have been deposited.

After a desired number of layers is deposited, the layered structure may be separated into a plurality of drug delivery units. For example, the layered structure may be cut in a direction or directions perpendicular to the planes of the layers to form rectangular or cubic drug delivery units. The size of the drug delivery units may be based, for example, on the desired drug release kinetics and/or shape performance. As used herein, “shape performance” refers to the types and forms of motion which a drug delivery unit may or is likely to undergo when administered to a patient. For example, a rectangular delivery unit having one dimension longer than the other two may be more likely to become lodged in openings within a vessel compared to a spherical delivery unit. The drug delivery composition may also be placed on the surface of a drug delivery device. The composition may be deposited via nanofabrication processes directly onto a device, or the composition may be pre-fabricated via a nanofabrication process and then placed on a device.

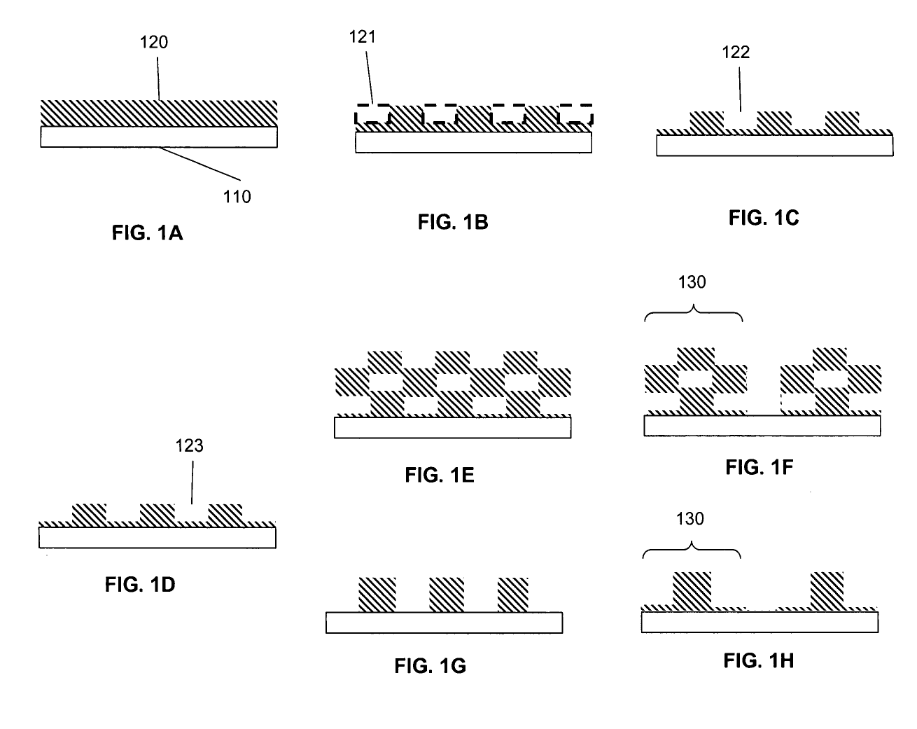

FIG. 1 shows a general method of manufacturing a drug delivery composition using a nanofabrication process. In FIG. 1A, a first layer of matrix material 120 is provided, for example, by depositing matrix material 120 on substrate 110. First layer of matrix material 120 may comprise a biodegradable or biostable polymer, a synthetic or natural material, a metal, or other suitable material. The matrix material may be chosen based on the therapeutic agent to be delivered, or it may be chosen for other properties. Substrate 110 may be any suitable material. It may be advantageous to use a material present in matrix layer 120 or a material similar or complementary to the matrix material, such that substrate 110 may be part of the delivery composition. For example, if matrix material 120 is a biodegradable polymer, it may be desirable for substrate 110 to comprise a biodegradable polymer. It may similarly be advantageous for substrate 110 to comprise a material from which matrix layer 120 may be easily removed. Substrate 110 may also comprise a medical delivery device body, such as a stent body, on which the delivery composition is placed. In an alternative embodiment, the process may be carried out without a substrate 110.

Referring to FIGS. 1B and 1C, pores 122 are formed in matrix layer 120. As shown in FIG. 1B, portions of matrix layer 120 may be marked or otherwise indicated as desired locations 121 for pores to be formed. Matrix material may be removed from these locations. Alternatively, matrix material in the desired locations 121 may be depressed so as to form pores. FIG. 1C shows matrix layer 120 having pores 122. It may be desirable for pores 122 to be roughly rectangular. As used herein, a pore is “rectangular” if it has roughly perpendicular sides and bottom, such that it is roughly in the shape formed by a rectangle extruded in a direction perpendicular to the plane of the rectangle, i.e., a rectangular solid. After pores 122 have been formed in matrix layer 120, a therapeutic agent 123 is placed into pores 122 as shown in FIG. 1D. The therapeutic agent may be deposited via a nanolithographic process or other similar means, or it may be placed using other methods. Other layers, as described below, may be provided prior to placing therapeutic agent 123 in pores 122.

The steps described with respect to FIGS. 1A-1D may be repeated, such that additional layers of matrix material and therapeutic agent are provided on the first layer of matrix material 120, with pores being formed in each layer. The pores may be formed such that they are not in registration with pores in a lower layer, such as is shown in FIG. 1E. Alternatively, the pores may be formed such that each pore is in registration with a pore immediately below it as shown in FIG. 1G. The position of pores in adjacent layers may be determined based on the desired release rate of the therapeutic agent. For example, in configurations where each pore is in registration with a pore immediately below it, fewer delivery outlets will be available in the composition than in configurations where each pore is not in registration with a pore immediately below it. However, such configurations will have delivery outlets extending further into the matrix material and containing more therapeutic agent.

As mentioned above, a therapeutic agent may be placed in the pores of each layer prior to providing the following layer, or it may be placed into the pores of all layers after the desired number of layers have been provided. If more than one therapeutic agent is desired, each therapeutic agent may be placed into the pores of each appropriate layer at any time after the layer has been provided and pores have been formed in the layer. The same or different therapeutic agents can be placed in different pores and/or different layers of matrix material.

After a structure comprising the desired number of layers with pores is provided, the structure may be divided into drug delivery units as shown in FIGS. 1F and 1H by cutting or otherwise separating portions of the layers. The precise size and shape of the drug delivery units may be determined based on the desired release rate, kinetics, and shape performance, of the drug delivery units, and other factors. It may be preferred to separate the layers into drug delivery units such that the drug delivery units are rectangular or cubic in order to achieve desired release rates and kinetics, and/or desired shape performance. For example, it may be preferable to calculate release rates based on cubical drug delivery units with each drug delivery unit having a known number of pores, in order to simplify calculations of dosage. Similarly, if drug delivery units are desired that may be capable of blocking an opening at a desired treatment site, larger rectangular drug delivery units may be preferable.

The process as illustrated in FIGS. 1A-1H results in micro-cubic or micro-rectangular structures on the surface of a substrate, such as the body of an implantable medical device such as a stent. Alternatively, when carried out without a substrate, the process results in micro-cubic or micro-rectangular structures that may be employed elsewhere. For example, the structures may be suspended in a fluid for injection.

Embodiments of the invention allow for precise control over the pore structure and final size of drug delivery compositions and coatings. For example, the pore arrangement may be configured to result in specific release rates. The invention further allows for precise control at very small scales, such as allowing for pores on the order of nanometers. In some embodiments, pores with dimensions on the order of 100 nm may be created. For example, rectangular pores may be created that have one dimension of about 100 nm parallel to the layer in which the pores are created. In some embodiments, each pore will have a diameter of not more than about 100 nm, where the “diameter” of a pore is the longest distance measurable across the pore in a direction parallel to the layer in which the pore is created. The pores may be all the same size and shape, or they have varying sizes and shapes. Such variation may be desirable, for example, to achieve release rates that vary with time. In some embodiments, the pores may have an average diameter of not more than about 100 nm.

Different numbers and thicknesses of layers may be used to create layered structures of the desired dimensions. Layers may be created as thin as about 1 nm, or as thick as several microns. The number and thickness of deposited layers may be determined based on desired release rates. Similarly, when a layered structure is separated into drug delivery units, the characteristics of the units may be further controlled by varying the size of the units. In some embodiments, drug delivery units with dimensions of about 100-500 μm may be created. Other pore sizes and drug delivery unit sizes are possible.

In an exemplary nanofabrication method according to the invention, reverse imprinting or soft lithography may be used to manufacture a drug delivery composition. A stamp may be coated with a polymer material or other matrix material. When the stamp is removed, a layer of the matrix material may be deposited and remain on the substrate. The matrix material will have the “negative” image of the stamp, such that protrusions on the stamp result in depressions in the matrix material. Thus, pores may be created in a layer of matrix material with precise control of the size and shape of the pores. After the layer of matrix material is deposited, a therapeutic agent may be placed into the pores. The therapeutic agent may be placed into the pores using the same or a similar procedure as that used to deposit the matrix layer. For example, the therapeutic agent may be placed onto a stamp having the negative image of the first stamp, such that when the stamp is pressed onto the previously-deposited matrix later the therapeutic agent is transferred to the pores of matrix layer. The therapeutic agent may also be placed into the pores of the matrix layer using other means.

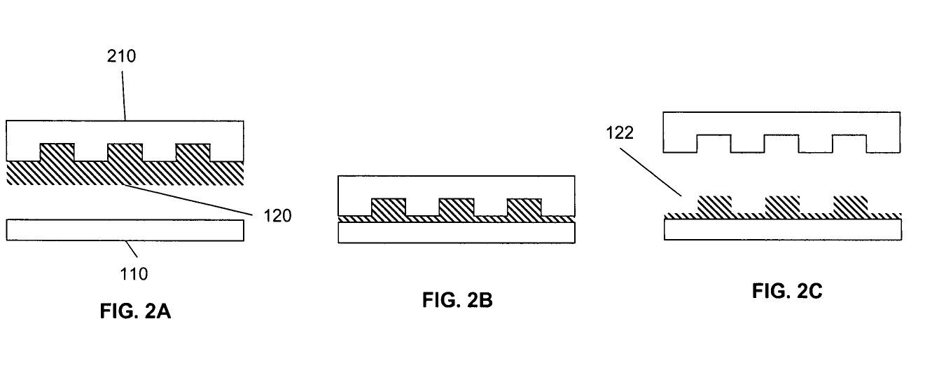

For example, FIG. 2 shows an exemplary method of forming pores in a layer of matrix material using reverse imprinting or soft lithography, which can be used to manufacture a drug delivery composition. In FIG. 2A, substrate 110 is provided as previously described, although the process may be carried out without a substrate. A matrix material 120 is placed on stamp 210 by any suitable process, such as spin-coating. Stamp 210 may comprise a metal, a polymer, or other suitable material. For some designs, it may be preferable to use a rigid stamp such as a metal stamp. In other cases, it may be preferable to employ soft lithography using a “soft stamp,” that is, a stamp that may deform when pressure is applied. Soft stamps may comprise an elastomeric material. Use of a soft stamp may be advantageous depending on the nature of the matrix material used in a specific configuration. For example, where high pressure may result in damage to the matrix material, a soft stamp may be preferred. Stamp 210 is then placed on substrate 110 to form a first layer of matrix material 120 and pressure is applied, as shown in FIG. 2B. The amount of pressure applied and the duration for which it is applied may vary depending on the matrix material being used. It may be preferred to treat substrate 110, matrix material 120, or both prior to placing matrix material 120 on the substrate in order to aid in deposition. For example, an adhesive may be applied. After matrix layer 120 has been placed on substrate 110 and pressure applied, stamp 210 is removed. Pores 122 are formed in matrix material 120 as shown in FIG. 2C. The depth of pores 122 may be controlled by controlling the pressure applied during the step illustrated in 2B. Matrix material 120 may be depressed in the locations corresponding to raised features of stamp 210.

After pores 122 are formed in matrix layer 120, the process may be repeated by providing layers placed on the first matrix layer 120 as described above. A therapeutic agent is then placed in the pores and the layers may be separated into drug delivery units as previously described and as illustrated in FIGS. 1D-1H.

Another exemplary method of manufacturing a drug delivery composition comprises nanoimprinting. In this method, a layer of matrix material as previously described may be deposited on a substrate. A stamp or mold may then be placed on the layer of matrix material. In some cases it may be preferable to use an elastomeric stamp. Elastomaeric stamps may allow for matrix material to be deposited at a lower pressure than those achievable using a rigid stamp. If an elastomeric stamp is used, the process may be referred to as soft lithography. When pressure is applied, the matrix material may be deformed in the shape of the stamp or mold. Such a process may be used to create pores of precise size and shape in the matrix material. The stamp is then removed, resulting in a layer of matrix material with pores. It may be preferable for the pores to be in the shape of rectangular solids as previously described. Therapeutic agent may then be placed in the pores.

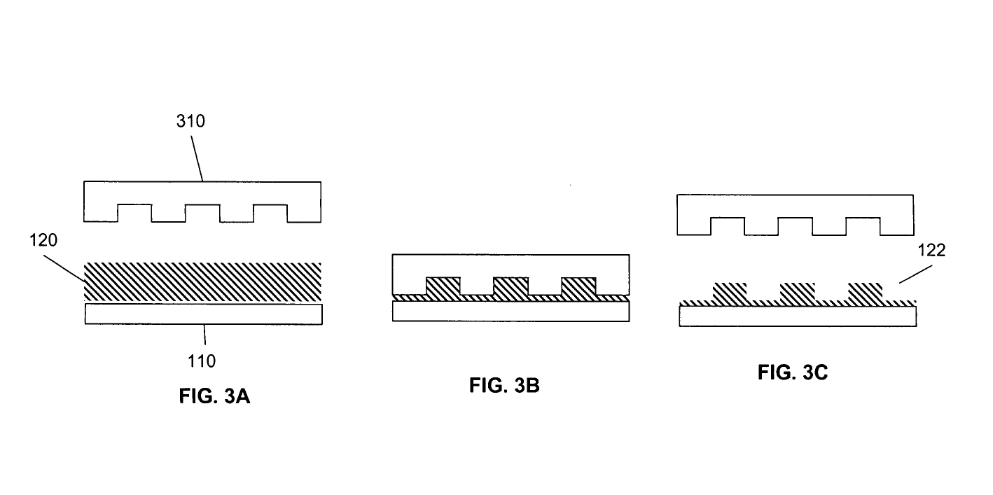

For example, FIG. 3 shows an exemplary method of forming pores in a layer of matrix material using nanoimprinting, which can be used to manufacture a drug delivery composition. Matrix layer 120 and substrate 110 are provided as previously described, although the process may be carried out without a substrate. Stamp 310 may be a rigid stamp, for example a stamp comprising metals or metallic compounds, or it may be a soft stamp. Soft stamps may comprise an elastomeric material, for example poly(dimethylsiloxane) (PDMS). Soft stamps may be desirable for use with some materials, as less pressure may be required and the stamp may be more easily fabricated. Stamp 310 may be fabricated using methods known in the art, for example by curing a layer of PDMS against a lithographically prepared master. Stamp 310 is placed on matrix layer 120, and pressure is applied as shown in FIG. 3B. The amount and duration of pressure may be determined based on the matrix material used for matrix layer 120, the desired pore depth, and other factors. Stamp 310 is removed as shown in FIG. 3C. Portions of matrix layer 120 are depressed in locations corresponding to raised features of stamp 310, forming pores 122 in matrix layer 120.

As previously described and as illustrated in FIGS. 1D-1H, the process illustrated in FIG. 3 may be repeated so as to form multiple layers of matrix material. Therapeutic agent may be placed in pores 120, and the layered structure may be separated into delivery units.

Another method of manufacturing a drug delivery composition comprises nanolithography or photolithography. A layer of matrix material is obtained, which optionally may be deposited on a substrate. The matrix material may comprise a resist material, such that the material is altered when exposed to a specific wavelength of radiation. A mask is created from a material which is opaque to radiation of at least the wavelength to which the matrix material is reactive. The mask may be constructed such that it is a two-dimensional representation of the structure to be formed in the matrix material. That is, the mask may be a sheet with length and width of at least the length and width of the matrix layer, with voids in locations corresponding to desired pore locations. Similarly, the mask may comprise voids corresponding to locations where non-pore matrix material is desired.

The mask may be placed on the matrix layer, such that the matrix layer is covered by the mask. Radiation may then be directed at the top of the mask, so as to be incident on the mask and any portion of the matrix layer exposed by the mask. After exposure to the radiation, the mask may be removed. The matrix material may then be treated with, for example, a solvent, so as to remove either the exposed portion of the matrix material or the unexposed portion so as to form a matrix layer having pores. A therapeutic agent may then be placed in the pores of the matrix layer. The process may be repeated as previously described in order to form a delivery composition having multiple layers.

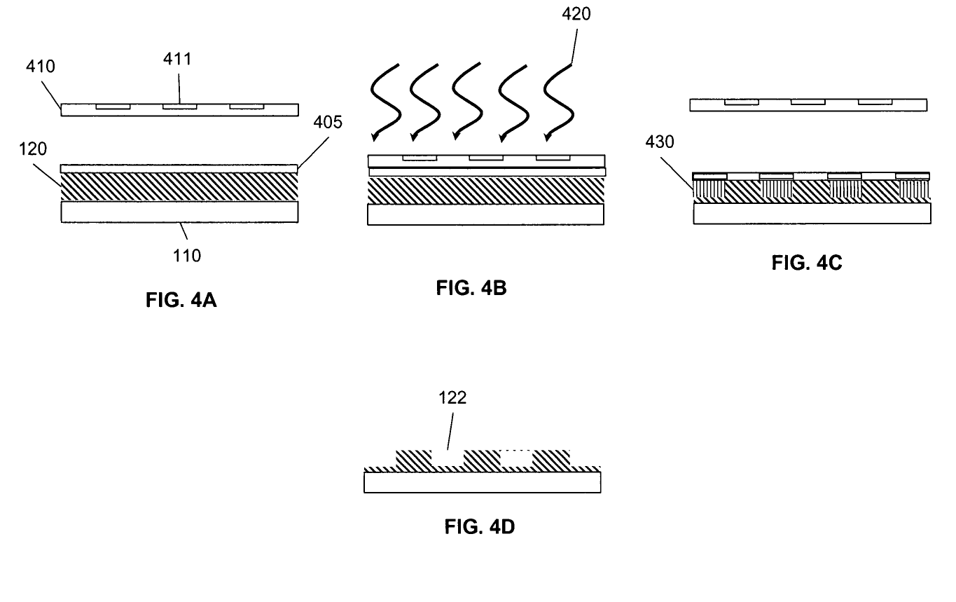

For example, FIG. 4 shows an exemplary method of forming pores in a layer of matrix material using nanolithography, which may be used to manufacture a drug delivery composition. Matrix layer 120 and substrate 110 are provided as previously described, although the process may be carried out without a substrate. Resist layer 405 may be placed on matrix layer 120. If present, resist layer 405 should comprise a photoactive material; that is, a material that becomes more or less soluble when exposed to a certain wavelength of radiation. If a resist layer is not present, matrix layer 120 should comprise a photoactive material. Mask 410 with openings 411 is placed over matrix layer 120. Radiation of the wavelength to which matrix layer 120 or resist layer 405 is photosensitive is directed at mask 410, as shown in FIG. 4B. The mask is then removed. Regarding FIG. 4C, regions 430 of matrix layer 120 or resist layer 405 exposed to radiation by mask 410 should then be developed; that is, the regions are more or less soluble than regions not exposed to radiation. Matrix layer 120 and, if present, resist layer 405 is then washed with a solvent. Regions 430 of the matrix layer 120 or, if present, the resist layer 405, are removed by the solvent. Alternatively, unexposed portions of the photoactive layers may be removed depending on the material used to form the matrix or resist layer, the radiation used, and the solvent used. Pores 122, as shown in FIG. 4D, are formed in the matrix layer when exposed material is removed. Similarly, pores may be formed by removing unexposed material. If a resist layer is used, pores may be formed by removing exposed or unexposed resist layer material and matrix layer material immediately below the removed portions of the resist layer. Remaining portions of the resist layer, if present, may be removed in a separate process.

As previously described and as illustrated in FIGS. 1D-1H, the process illustrated in FIG. 4 may be repeated so as to form multiple layers of matrix material. Therapeutic agent may be placed in pores 120, and the layered structure may be separated into delivery units.

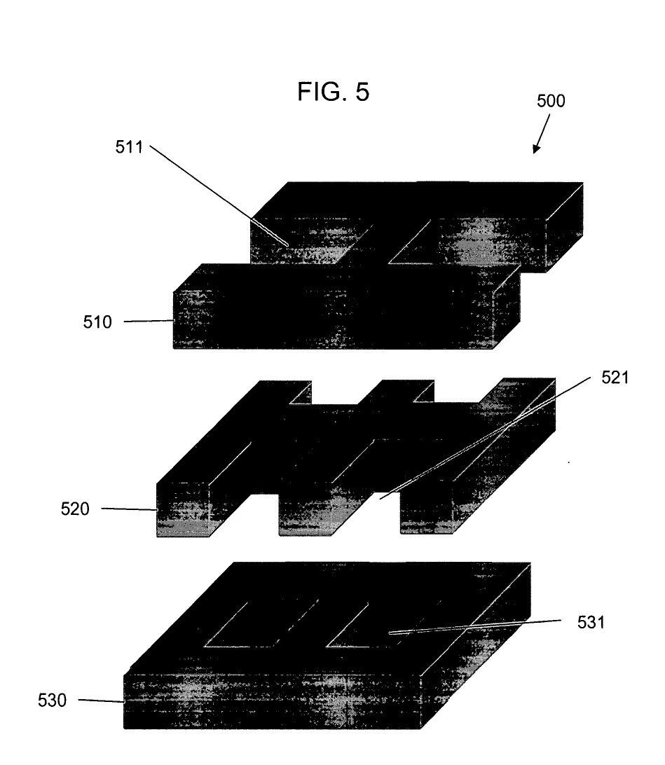

FIG. 5 shows an exploded perspective view of a delivery unit. Delivery unit 500 comprises layers 510, 520, and 530. Each layer may be disposed directly adjacent to and in physical bonded contact with the layer immediately above, below, or both. Layers 510, 520, and 530 may be formed using a nanofabrication method as described. The layers are shown separated for illustration purposes. It will be understood that the layers and specific pore structures illustrated are examples only, and that other structures are within the scope of the invention. A therapeutic agent or agents may be placed into pores 511, 521, and 531. Therapeutic agent may be placed in the pores of each layer after the layer is fabricated, or therapeutic agent or agents may be placed into the pores of the completed structure. Delivery unit 500 may be rectangular, that is, it may have unequal length, width, and/or height. It may also be cubical such that it has equal length, width, and height. Cubical delivery units may be useful to allow for simplified modeling and manufacture of the drug delivery units and therapeutic uses.

In some embodiments, a drug delivery unit may comprise a lattice of matrix material and therapeutic agent. As used herein, a “lattice” refers to a three-dimensional structure having a regular, periodic arrangement. For example, a lattice may be formed using the above methods to provide multiple layers having pores of equal volume, where there is a volume of matrix material equal to the volume of one pore between any two adjacent pores. In some embodiments, there may be unequal volumes of matrix material and therapeutic agent. As used herein, “lattice spacing” or “pore spacing” will be understood to refer to the distance from the edge of a first pore to the nearest edge of an adjacent pore, measured across the width of the first pore. That is, lattice spacing generally measures the width of a pore and adjacent matrix material between the pore and an adjacent pore. Configurations utilizing a lattice structure may be preferable in order to provide precisely-known release rates and kinetics, for example by simplifying calculations necessary to predict release rates based on drug delivery unit shape and size.

Since embodiments of the present invention provide delivery compositions with controllable configurations and geometries, it may be desirable to manufacture the compositions or delivery units based on desired release rates, release kinetics, and/or shape performance. Release rates and kinetics may be determined by the properties of the therapeutic agent to be delivered, properties of the disease to be treated, or other factors. The desired structure for a delivery composition or drug delivery unit may be determined based on the desired shape performance. A desired structure for the delivery composition or unit may also be based on the desired release rate. For example, if a high release rate is desired, a structure having larger, more shallow pores may be advantageous in order to allow for rapid diffusion of the therapeutic agent after administration to a patient. A delivery composition may be manufactured using nanolithographic processes to provide one or more layers of matrix and therapeutic material as previously described. The delivery composition may then be formed into drug delivery units and administered directly, or it may be incorporated into other delivery devices such as implantable medical devices.

The therapeutic agent used in the present invention may be any pharmaceutically acceptable agent such as a non-genetic therapeutic agent, a biomolecule, a small molecule, or cells. Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor \ddot{γ} and \ddot{γ}, platelet-derived endothelial growth factor, platelet-derived growth factor, tumor necrosis factor \ddot{γ}, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin−) cells including Lin− CD34−, Lin−CD34+, Lin−cKit+, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible and any of the embodiments of the present invention can comprise multiple therapeutic agents that are the same or different.

The polymers used in the matrix layers of the present invention may be biodegradable or non-biodegradable. Non-limiting examples of suitable non-biodegradable polymers include polystrene; polyisobutylene copolymers, styrene-isobutylene block copolymers such as styrene-isobutylene-styrene tri-block copolymers (SIBS) and other block copolymers such as styrene-ethylene/butylene-styrene (SEBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Any embodiments of a medical device of the present invention may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Non-limiting examples of medical devices according to the present invention include catheters, guide wires, balloons, filters (e.g., vena cava filters), stents, stent grafts, vascular grafts, intraluminal paving systems, implants and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like.

One of skill in the art will realize that the embodiments described and illustrated herein are merely illustrative, as numerous other embodiments may be implemented without departing from the spirit and scope of the present invention. Moreover, while certain features of the invention may be shown on only certain embodiments or configurations, these features may be exchanged, added, and removed from and between the various embodiments or configurations while remaining within the scope of the invention. Likewise, methods described and disclosed may also be performed in various sequences, with some or all of the disclosed steps being performed in a different order than described while still remaining within the spirit and scope of the present invention.

1. A method of manufacturing a drug delivery composition, comprising the steps of:
providing a medical device;
providing a first layer of matrix material on the medical device;
forming pores in the first layer of matrix material by nanofabrication;
providing a second layer of matrix material on the first layer of matrix material;
forming pores in the second layer of matrix material by nanofabrication; and
placing a therapeutic agent in the pores of the first and second layers of matrix material.

2. (canceled)

3. The method of claim 1 wherein the medical device is a stent.

4. The method of claim 1 wherein the medical device comprises the same material as the first layer of matrix material.

5. The method of claim 1 wherein the pores have a rectangular cross-section.

6. The method of claim 1 wherein each of the pores has a diameter of about 100 nm.

7. The method of claim 1 wherein the pores have an average diameter of not more than about 100 nm.

8. The method of claim 1 wherein the drug delivery composition is about 100 μm to about 500 μm thick.

9. The method of claim 1 wherein the pores of the second layer of matrix material are in registration with the pores of the first layer of matrix material.

10. The method of claim 1 wherein at least one of the steps of forming pores is performed via nanoimprinting.

11. The method of claim 1 wherein at least one of the steps of forming pores is performed via reverse imprinting.

12. The method of claim 1 wherein at least one of the steps of forming pores is performed via nanolithography.

13. The method of claim 1 wherein at least one of the steps of forming pores is performed via photolithography.

14. The method of claim 1 further comprising providing a third layer of matrix material on the second layer of matrix material.

15. The method of claim 1 wherein the therapeutic agents are placed into the pores of each layer prior to providing a subsequent layer.

16. The method of claim 1 further comprising the step of:
cutting the layers into drug delivery units in a direction perpendicular to the planes of the first and second layers to achieve a desired release rate.

17. The method of claim 16 wherein the drug delivery units are cubical.

18. The method of claim 16 wherein the drug delivery units have a diameter of about 100-500 μm.

19. The method of claim 17 wherein the drug delivery units have sides of not more than about 100-500 μm.

20. The method of claim 1 wherein the structure of the pores comprises a lattice.

21. The method of claim 1 wherein the position of the pores in adjacent layers is determined by the desired release kinetics.

22. The method of claim 1 wherein the structure of the composition is based on a desired shape performance.

23. The method of claim 1 wherein the structure of the composition is based on a desired release rate.

24. A method of manufacturing a drug delivery device, comprising:
providing a medical device body;
depositing a first layer of matrix material on the medical device body;
forming pores with a rectangular cross-section in the first layer of matrix material by nanofabrication; and
depositing therapeutic agents in the pores of the first layer of matrix material.

25. The method of claim 24 wherein the medical device is a stent.

26. The method of claim 24 wherein the pores are formed by a nanofabrication process selected from the group consisting of nanoimprinting, reverse imprinting, nanolithography, and photolithography.

27. The method of claim 24, further comprising:
depositing a second layer of matrix material having rectangular pores on the first layer of matrix material; and
placing a therapeutic agent in the pores of the second layer of matrix material.

28. A medical device comprising:
a medical device body;
a plurality of layers of a matrix material disposed on the medical device body, each layer having rectangular pores; and
a therapeutic agent;
wherein the therapeutic agent is disposed in the pores of each layer of matrix material.

29. The device of claim 28 wherein the medical device is a stent.

30. The method of claim 28 wherein the pores are formed by a nanofabrication process selected from the group consisting of nanoimprinting, reverse imprinting, nanolithography, and photolithography.
